(19) United States
(12) Patent Application Publication
(10) Pub. No.: US 2016/0278830 A1
(43) Pub. Date: Sep. 29, 2016

(54) REINFORCED CANNULATED IMPLANT SYSTEM AND METHOD

(71) Applicant: William ARRINGTON, Mesquite, TX (US)

(72) Inventor: William ARRINGTON, Mesquite, TX (US)

(21) Appl. No.: 14/668,554

(22) Filed: Mar. 25, 2015

(51) Int. Cl. A61B 17/86

(52) U.S. Cl. CPC A61B 17/864; A61B 17/8615; A61B 17/8625; A61B 17/8888

(57) ABSTRACT

A cannulated implant for bone fixation is reinforced by inserting a rigid support core into the cannulated implant's cavity to provide a strengthened cannulated system, better mimicking the strength of an implant having a solid length. The rigid support core sits flush against the inner walls of the implant's cavity, avoiding any disruption with any driver placement, and is fixated to the implant by screwing, tamping into place, or by taper cold fusion.

### TECHNICAL FIELD

The present invention generally relates to a reinforced cannulated implant system for the fixation of bones. More specifically, a cannulated implant system having a strengthening dowel inserted therein, decreasing the likelihood of mechanical failure.

### BACKGROUND OF THE INVENTION

Many surgical implants are implanted using a cannulated system. Cannulated systems are screws or rods that have a hollow central shaft, which can be placed around a guide wire or guide pin. Guide pins have a much smaller diameter than the screw or rod implant, allowing for a surgeon's precise placement of the guide pin into bone with minimal trauma. Once the guide wire is desirably placed across an interface between two bone structures, the hollow screw or rod is implanted over and around the guide wire and into the bone. The guide wire is then removed, leaving an open void in the center, or a hollow core implant.

The screws or rods are often fixed across joints or fractured bones to provide stability and rigidity. However, the bending and shear strength of cannulated systems versus solid core systems is compromised for the benefit of ease and precision of the placement and insertion of the screw or rod implant. The hollow core of the cannulated system can cause increased instances of mechanical failure due to shear and bending forces. This leads to deformation or breakage of the screw or rod. For the most part, when the screw or rod fails, this leads to a follow-up surgery for removal of the fractured hardware. When the hardware is fractured, the proximal part of the hardware can be removed but the distal end of the hardware is usually implanted deep into the bone and is not retrievable. This leaves a deep, unstable piece of hardware in a patient's body.

There are multiple surgical methods and different cannulated systems on the market that claim to ease the removal of fragmented screws and hardware due to mechanical failure of cannulated systems. Breakage of surgical implants can occur, often leading to additional surgeries or disabilities for patients. There is a need for cannulated systems capable of preserving and/or strengthening the hollow screws or rods, which are preferable for exact placement within the bone.

### SUMMARY

The present disclosure drastically reduces the frequency of cannulated medical implant failures by using a support core or rod within the implant's cavity. The support core is made with materials similar to that of the implants (screws, rods, nails, etc.) thereby providing increased rigidity and tensile strength similar to that of a solid core system. This stronger cannulated implant system leads to a decrease in mechanical failure of implants and a decrease in return of patients to the operating room for removal of failed hardware.

A reinforced cannulated system for bone fixation can comprise a screw or rod having a length that runs from a proximal head to a distal tip and a cavity that runs throughout the length between the proximal head and distal tip, said proximal head comprising a driver placement; and a rigid support core securable within the cavity, the rigid support core having an attaching surface on an exterior side for fastening to a corresponding inner surface of the cavity.

A method for reinforcing a cannulated system, said method comprising the steps of: inserting an elongated medical implant into a bone, the elongated medical implant comprising a proximal head opposite a distal tip and a cavity between the proximal head and distal tip; filling the cavity with a rigid support core; and securing the rigid support core within the cavity, said rigid support core firmly attached therein without disrupting any driver placement of the medical implant. Other steps for bone fixation may also be used in one aspect; including without limitation: cutting or drilling bone, placing a guide wire within the bone at a desired position, or removing the guide wire.

In one embodiment, the elongated length or elongated medical implant comprises a cylindrical shaft and the cavity is a lumen running axially through the implant from the proximal head to the distal tip, such as with a screw, rod or pin often used as a surgical implant for bone fixation. In one embodiment, the rigid support core comprises a size (i.e., length and width, or diameter) slightly smaller than that of the cavity or lumen, wherein slightly smaller means sized to fit tightly within the cavity or lumen. The rigid support core should sit flush against the inner walls of the cavity or lumen, avoiding any disruption of the driver placement.

The rigid support core is fixated to the hardware or implant by screwing, tamping into place, or by taper cold fusion. The support core will reinforce the cannulated system to help prevent mechanical failure or breakage against excessive tension, stress, and sheer forces. The reinforcing system for cannulated hardware in surgical cases described herein thereby increases the strength of the implant.

In one embodiment, a reinforced cannulated system for bone fixation comprises a cannulated implant comprising a length running between a proximal head and a distal tip, and an axial cavity running along the length; and a rigid support core configured to be secured within the axial cavity after the implant is placed into a bone structure, the rigid support core having an exterior attaching surface corresponding to an inner attaching surface of the cavity. In another embodiment, the rigid support core of any other embodiment described herein substantially fills the cavity. In another embodiment according to any other embodiment described herein the cannulated implant is a screw with external threading on at least a portion of the elongated length opposite the proximal head. In another embodiment according to any other embodiment described herein the exterior attaching surface on said rigid support core is threaded. In another embodiment according to any other embodiment described herein the rigid support core is a rod which is geometrically similar in shape to the axial cavity.

In another embodiment according to any other embodiment described herein the exterior attaching surface on said rigid core is a tapered head that abuts said inner attaching surface of the cavity, which is a resisting portion of the inner surface of the cavity. In another embodiment according to any other embodiment described herein the rigid support core comprises a driver head at one end and a neck portion between the driver head and the threaded exterior attaching surface, wherein the driver head and neck portion break away by application of a predetermined amount of torque to the driver head, wherein after the driver head and neck portion break away, the remaining rigid support core is below a driver placement on the proximal head of the implant.

In one embodiment, the invention comprises a support core for a cannulated medical implant comprising an exterior attaching surface corresponding to an inner attaching surface of a cavity of the cannulated medical implant, wherein the cannulated medical implant comprises a length running between a proximal head and a distal tip, wherein the cavity is an axial cavity running along the length, and wherein the support core is configured to be secured within the axial cavity after the implant is placed into a bone structure.

In one embodiment of the invention, a method for reinforcing a cannulated implant comprises the steps of inserting a cannulated implant into a bone, the cannulated implant comprising a length that runs between a proximal head and a distal tip, and an axial cavity that runs along the length of the implant; filling the cavity with a rigid support core; and securing the rigid support core within the cavity. In another embodiment according to any other embodiment described herein the rigid support core comprises a tapered end near the proximal head of the cannulated implant and wherein the securing step comprises tapping the tapered end into the cavity such that the rigid support core substantially fills the cavity.

In another embodiment according to any other embodiment described herein the rigid support core comprises an external attaching surface, and wherein said securing step comprises fastening the external attaching surface to a corresponding internal surface of the cavity. In another embodiment according to any other embodiment described herein the attaching surface is threaded to mate with threading on the corresponding internal surface of the cavity. In another embodiment according to any other embodiment described herein the rigid support core is one integral solid piece shaped to substantially fill the cavity. In still another embodiment according to any other embodiment described herein the rigid support core comprises a driver head to assist in filling the cavity, and a neck portion below the driver head, wherein the method comprises after the securing step: the step of applying torque to the neck portion with the driver head, thereby breaking off the neck portion and the driver head and leaving the rigid support core within the cavity.

Other aspects, embodiments and features of the invention will become apparent in the following written detailed description and accompanying drawing.

### BRIEF DESCRIPTION OF THE DRAWINGS

The novel features believed characteristic of the invention are set forth in the appended claims. The invention itself, however, as well as mode of use and advantages thereof, will best be understood by reference to the following detailed description of illustrative embodiments when read in conjunction with the accompanying drawings, wherein:

FIG. 1 is a perspective view of one embodiment of a cannulated implant described herein.

FIG. 2 is a cross-sectional view of the cannulated implant of FIG. 1.

FIG. 3 is a perspective view of one embodiment of a rigid support core described herein.

FIG. 4 is an exploded view of one embodiment of the cannulated system described herein.

FIG. 5 is a perspective view of another embodiment of a cannulated implant described herein.

FIG. 6 is a cross-sectional view of the cannulated implant of FIG. 5.

FIG. 7 is a perspective view of one embodiment of a rigid support core described herein.

FIG. 8 is an exploded view of one embodiment of the cannulated system described herein.

### DETAILED DESCRIPTION

Generally, the reinforced cannulated system for bone fixation comprises a cannulated implant having a length that runs between a proximal head and a distal tip, and an axial cavity running along said length, said proximal head comprising a driver placement; and a rigid support core securable within the cavity, the rigid support core having an attaching surface on an exterior side configured to fasten to a corresponding inner surface of the cavity. In one embodiment, the implant is tubular and the axial cavity is a lumen that runs from the proximal head to the distal tip, such as with a hollow screw or rod. The driver placement comprises an engagement portion configured to receive a driver used by a surgeon to place or screw the implant into a bone for fixation across two structures. For example, a screw driver can be used to turn a screw implant and thereby drive it into the bone structure, or a force driver can be used to translate force from a hammer to the implant and thereby drive it into the bone structure.

The rigid support core comprises a shape substantially identical to at least a portion of the cavity of the implant and a size sufficiently smaller than that of the cavity to fit tightly therein. The rigid support core within the cavity or lumen of the elongated length should comprise or consist of one solid unit. In one embodiment, the rigid support core comprises a length substantially equivalent to the length of the cavity or lumen.

As used herein, the term “rigid” means strong or stiff, in the sense that a rigid support core can be held between two fingers at one end, suspended in the air, and the support core will not deform under its own weight. The rigid support core is not easily bent or forced out of shape and remains generally inflexible within the surrounding walls of the implant. In one embodiment, the rigid support core comprises a material composition similar to that of the medical implant. Suitable material composition may comprise, by way of example, titanium alloy, stainless steel, or any medical-grade or surgical-grade metal, or physiologically absorbable material.

The implant may comprise a medical rod or screw (whether cortical or cancellous external threading, or partially or fully threaded) with any number of diameters or lengths used in the industry.

The method for reinforcing a cannulated system generally comprises inserting a cannulated medical implant into a bone structure, filling the cavity of the medical implant with a rigid support core, and securing the rigid support core within the cavity, the rigid support core firmly attached therein without interfering with any driver placement on the head of the implant. Other steps for proper placement of the medical implant may also be used as described above and as known in the art.

Embodiments of the reinforced cannulated system described above will now be explained with reference to the figures.

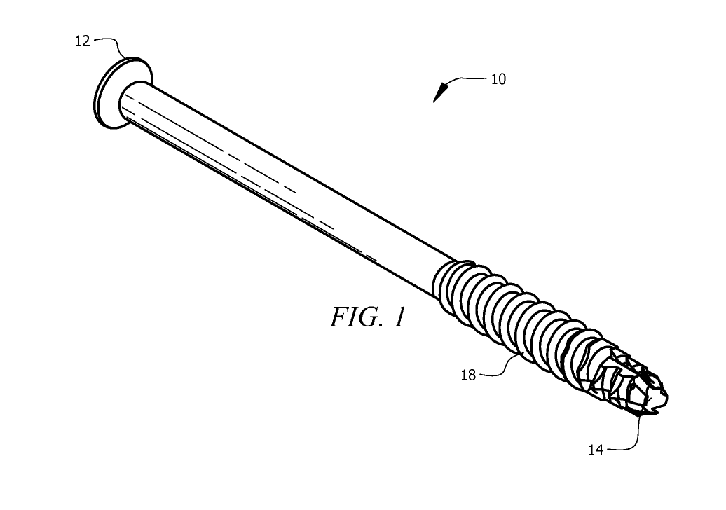

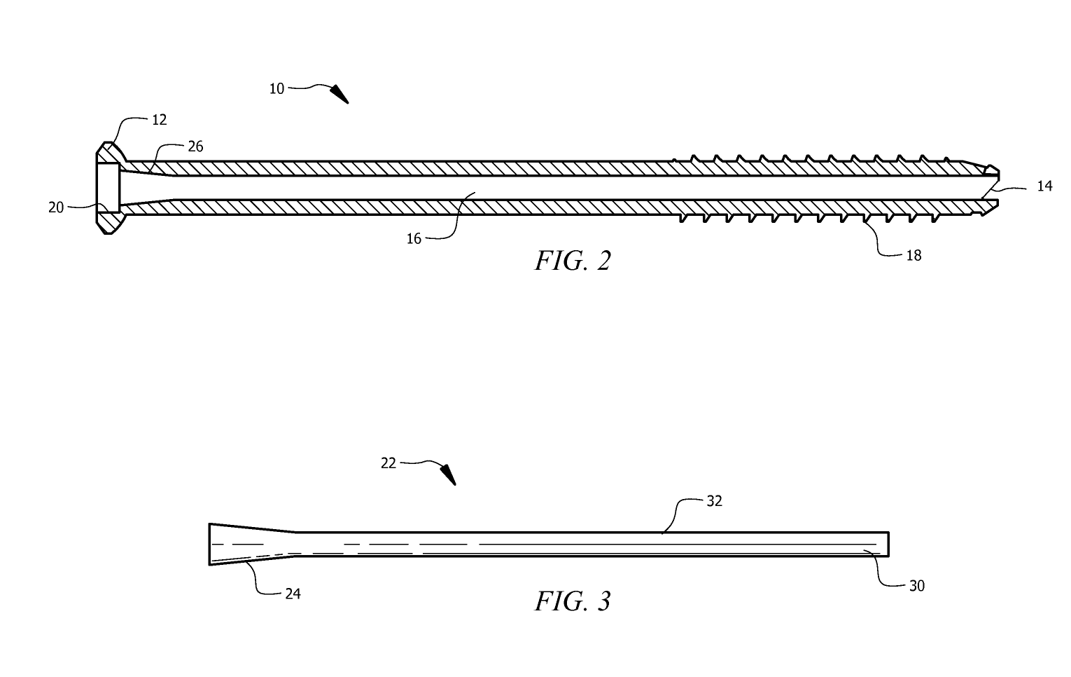

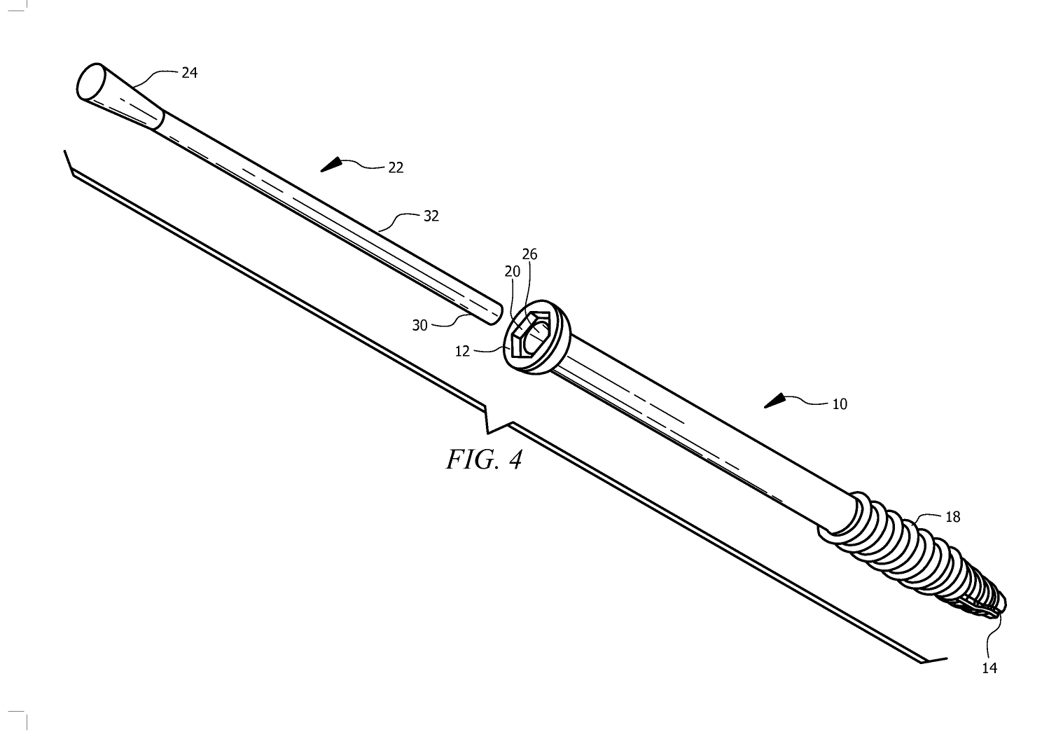

FIGS. 1 and 2 depict, respectively, perspective and cross-sectional views of one embodiment of a cannulated implant 10 for use in a strengthened cannulated system described herein. The implant 10 comprises a length running between a proximal head 12 and a distal tip 14 and an axial cavity 16 running along the length of the implant. The distal tip 14 may comprise a pointed or flat tip, depending on the intended use of the screw. In one embodiment, the length comprises external threading 18 near the distal tip 14. As perhaps best depicted in FIG. 4, the proximal head 12 comprises a driver placement 20 for engagement with a driver (not shown) used by a surgeon to screw the implant 10 into bone. The embodiment shown in FIG. 4 is a hexagonal driver placement. Below the driver placement 20, FIG. 2 depicts a tapered portion 26 of cavity 16. The tapered portion 26 corresponds to the tapered shape of the attaching surface 24 of support core 22, depicted in FIG. 3.

FIG. 3 depicts a support core 22 for insertion into the cavity 16 of the implant 10 depicted in FIG. 2. The support core 22 comprises a size and shape for substantially filling the cavity 16, with an attaching surface 24, a blunt tip 30, and a cylindrical solid shaft 32 extending between the attaching surface 24 and the blunt tip 30. The attaching surface 24 generally lies along an external portion of the support core 22. In the embodiment shown in FIG. 3, the attaching surface 24 lies near the proximal head 12 of the implant 10 when inserted therein. When the cannulated implant is tubular, the main shaft 32 of the support core 22 is cylindrical and the attaching surface 24 may extend around the circumference of the support core 22 on one end. As depicted in FIGS. 3 and 4, the attaching surface 24 of the support core 22 may comprise a tapered shape, or generally comprise a conical frustrum.

With reference to FIG. 4, when filling the cavity 16 with the rigid support core 22, the blunt tip 30 is first inserted into the cavity 16 through the opening at the proximal head 12 with a tamp type insertion, which may include pushing or tapping the core 22 into the cavity until the tapered end of the attaching surface 24 is forced within the cavity and beyond driver placement 20. Thus, the rigid support core 22 fits tightly within the cavity or lumen 16 without interfering with any driver placement used by a surgeon to secure the screw or implant 10 into a bone structure. This will allow the core 22 to sit flush with the proximal shaft opening of the cavity 16 avoiding any disruption of the driver placement. This allows for removal of the implant 10 (along with the support core 22) to remain possible. In one embodiment, the rigid support core 22 and the medical implant 10 are made of the same or like materials.

Although the implant depicted in the embodiments shown in FIGS. 1-4 contains a cavity that is non-cylindrical, in another embodiment of the present invention, the cavity inside the medical implant is substantially cylindrical. In this embodiment, the cavity can be substantially cylindrical, but the support core is not. For example, a support core with a very slight taper, wherein the maximum diameter of the support core at the large end of the taper is approximately the same diameter as the cavity or slightly larger, could be used. As such the attaching surface would be that portion of the support core that is used to affix a non-cylindrical support core within a substantially cylindrical medical implant cavity. The support core in this embodiment would be affixed within the cavity by a force relationship between the core and the implant at the attaching surface. This embodiment would be useful to provide a support core by itself that could be used in conjunction with existing cannulated medical implants, which typically use a cylindrical or substantially cylindrical cavity.

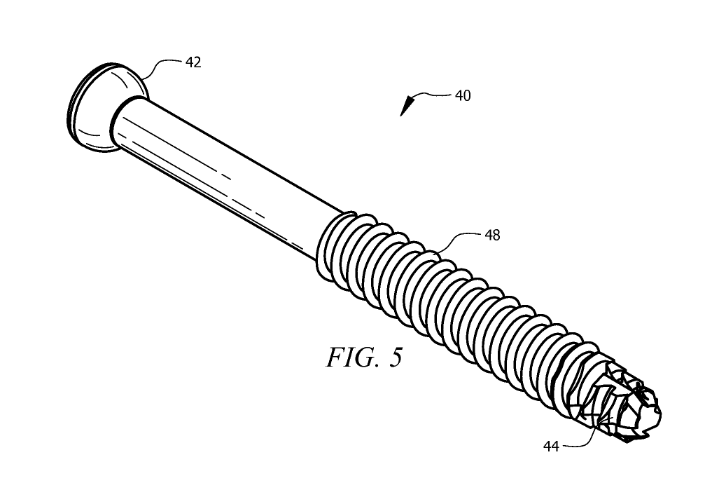

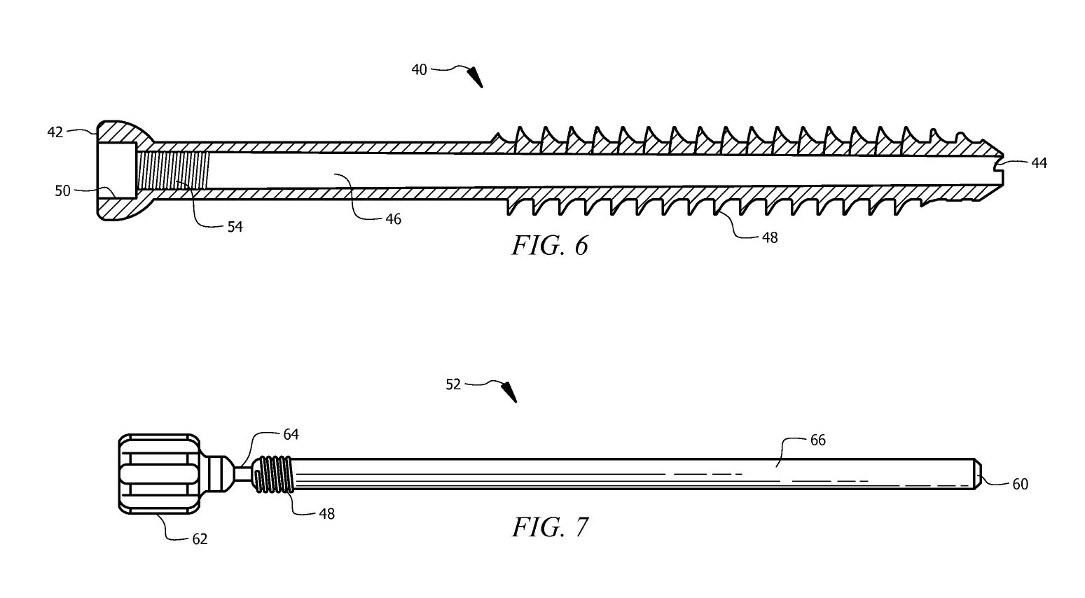

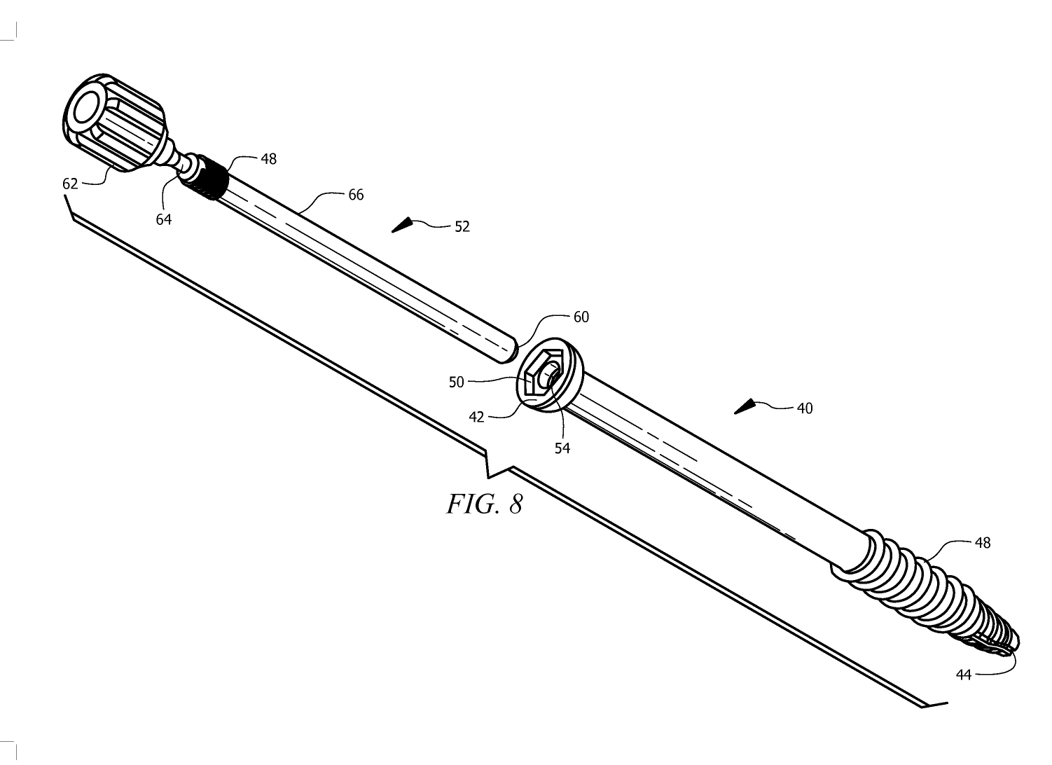

FIGS. 5 and 6 depict, respectively, perspective and cross sectional views of another embodiment of a cannulated implant 40 for use in a strengthened cannulated system described herein. The implant 40 comprises a length that runs between a proximal head 42 and a distal tip 44 and an axial cavity 46 extending between the proximal head 42 and distal tip 44. The distal tip 44 may comprise a pointed or flat tip, depending on the intended use of the screw. In one embodiment, the implant comprises external threading 48 near the distal tip 44. As perhaps best depicted in FIG. 8, the proximal head 42 comprises a driver placement 50 for engagement with a driver (not shown) used by a surgeon to screw in the implant 40 into a bone structure. The embodiment shown in FIG. 8 uses a hexagonal driver placement.

FIG. 7 depicts a support core 52 for insertion into the cavity 46 of the implant 40. The support core 52 comprises a size and shape for substantially filling the cavity 46, with an attaching surface 48, a blunt tip 60, and a cylindrical solid shaft 66 extending between the attaching surface 48 and tip 60. The support core 52 further comprises a driver head 62 and a neck portion 64 on the end opposite the blunt tip 60. The neck portion 64 is located between the driver head 62 and the attaching surface 48, forming a break-off support core. The attaching surface 48 lies along a portion of the support core 52 near the driver head. More specifically, in this embodiment, the attaching surface 48 lies on an end of the support core 52 corresponding to the end closest to the proximal head 42 when inserted within the implant's cavity 46. In the embodiment shown in FIGS. 7 and 8, the attaching surface 48 surrounds the circumference of the support core 52 on one end. In one embodiment, the attaching surface 48 of the support core 52 comprises external threading on the support core. The threading may surround one end of the support core 52 in one embodiment. When the attaching surface 48 comprises external threading on the support core 52, the implant cavity 46 comprises internal threading 54 that corresponds to the depth and spacing of the external threading 48 on the support core, such that the support core 52 can be engaged with the implant cavity by a turning motion.

With reference to FIG. 8, when filling the cavity 46 with the rigid support core 52, the blunt tip 60 is first inserted into the cavity 46 through the proximal head 42, and the support core 52 is slid or pushed into the cavity 46 until the attaching surface 48 is within the cavity 46. In one embodiment, the support core 52 may then be secured within the cavity by engaging the threaded portion of the attaching surface 48 with a corresponding internal threaded portion 54 of the cavity 46 of the implant 40. The driver head 62 can be used to engage and interlock the threaded portions of the cavity and support core. It should be understood that when the support core 52 comprises a male threading at the attaching surface 48, the adjacent internal surface 54 of the implant 40 will comprise a female threading for securing the support core 52 within the cavity 46. Likewise, if the support core 52 comprises female threading, the corresponding internal surface of the implant 40 will comprise male threading. In one embodiment, the attaching surface is a male/female threaded connection.

Once the threaded portions are engaged and the support core 52 is secured within the cavity 46, sufficient torque may be applied to the neck portion 64 through the driver head 62 to cause the driver head 62 to break off in the vicinity of the neck portion, leaving rigid support core 52 below a driver placement 50 on the proximal head 42 of the implant 40. Thus, the rigid support core 52 is situated tightly within the cavity or lumen without interfering with any driver engaging structures, which are needed to secure and/or remove the screw or implant 40 into a bone structure. If the implant requires removal for any reason, the support core 52 is removed along with the implant that contains the support core. In one embodiment, the rigid support core 52 and the implant 40 are made of the same or like materials. The embodiment shown in FIGS. 5-8 is more ideal for use in smaller cannulated systems due to the thickness of the wall of the head and neck of a smaller cannulated system.

The support core of the cannulated system as described herein reduces the risk of failure of the implant. By reinforcing cannulated systems as described herein with the filling of the implant's cavity below the driver placement, failure of cannulated systems after placement within the bone is prevented. The cannulated system would have similar tensile and shear strength as a solid core system. The cold fusion, tapered, fractioned, or threaded patterns of the support core that match the shape or pattern found within a hollow implant will resist the pull out and/or failure of the internal support core. The core and cannulated implant form one strong, integral unit within the bone, providing a cannulated system that will have a decreased risk of mechanical failure due to sheer, tension, stress, and bending. Also, if the implant needs to be removed for any reason, the core support will be removed along with the cannulated implant.

The invention illustratively disclosed herein suitably may be practiced in the absence of any element or limitation not specifically disclosed herein. The description of the present invention has been presented for purposes of illustration and description, and is not intended to be exhaustive or limited to the invention in the form disclosed. Many modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated. It will be understood by one of ordinary skill in the art that numerous variations will be possible to the disclosed embodiments without going outside the scope of the invention as disclosed in the claims.

I claim:

1. A reinforced cannulated system for bone fixation comprising:
a cannulated implant comprising a length running between a proximal head and a distal tip, and an axial cavity running along the length; and
a rigid support core configured to be secured within the axial cavity after the implant is placed into a bone structure, the rigid support core having an exterior attaching surface corresponding to an inner attaching surface of the cavity.

2. The reinforced cannulated system of claim 1 wherein the rigid support core substantially fills the cavity.

3. The reinforced cannulated system of claim 1 wherein the cannulated implant is a screw with external threading on at least a portion of the elongated length opposite the proximal head.

4. The reinforced cannulated system of claim 1 wherein the exterior attaching surface on said rigid support core is threaded.

5. The reinforced cannulated system of claim 1 wherein the exterior attaching surface on said rigid core is a tapered head that abuts said inner attaching surface of the cavity, which is a resisting portion of the inner surface of the cavity.

6. The reinforced cannulated system of claim 4 wherein the rigid support core comprises a driver head at one end and a neck portion between the driver head and the threaded exterior attaching surface, wherein the driver head and neck portion break away by application of a predetermined amount of torque to the driver head, wherein after the driver head and neck portion break away, the remaining rigid support core is below a driver placement on the proximal head of the implant.

7. The reinforced cannulated system of claim 1 wherein the rigid support core is a rod which is geometrically similar in shape to the axial cavity.

8. A support core for a cannulated medical implant comprising an exterior attaching surface corresponding to an inner attaching surface of a cavity of the cannulated medical implant, wherein the cannulated medical implant comprises a length running between a proximal head and a distal tip, wherein the cavity is an axial cavity running along the length, and wherein the support core is configured to be secured within the axial cavity after the implant is placed into a bone structure.

9. A method for reinforcing a cannulated implant, said method comprising the steps of:
inserting a cannulated implant into a bone, the cannulated implant comprising a length that runs between a proximal head and a distal tip, and an axial cavity that runs along the length of the implant;
filling the cavity with a rigid support core; and
securing the rigid support core within the cavity.

10. The method of claim 9 wherein the rigid support core comprises a tapered end near the proximal head of the cannulated implant and wherein the securing step comprises tapping the tapered end into the cavity such that the rigid support core substantially fills the cavity.

11. The method of claim 9 wherein the rigid support core comprises an external attaching surface, and wherein said securing step comprises fastening the external attaching surface to a corresponding internal surface of the cavity.

12. The method of claim 11 wherein the attaching surface is threaded to mate with threading on the corresponding internal surface of the cavity.

13. The method of claim 9 wherein the rigid support core is one integral solid piece shaped to substantially fill the cavity.

14. The method of claim 9 wherein the rigid support core comprises a driver head to assist in filling the cavity, and a neck portion below the driver head, wherein the method comprises after the securing step: the step of applying torque to the neck portion with the driver head, thereby breaking off the neck portion and the driver head and leaving the rigid support core within the cavity.
